(19) United States
(12) Patent Application Publication
(10) Pub. No.: US 2023/0086706 A1
(43) Pub. Date: Mar. 23, 2023

(54) Compounds for promoting follicle maturation

(71) Applicant: Aarhus Universitet, Aarhus C (DK)

(72) Inventors: Karin Lykke-Hartmann, Egå (DK); Gary Newton, London (GB); Duncan Hannah, Suffolk (GB); Robert Downham, Cambridgeshire (GB)

(21) Appl. No.: 17/789,310

(22) PCT Filed: Jan. 8, 2021
(86) PCT No.: PCT/EP2021/050262

§ 371 Date: Jun. 27, 2022

(30) Foreign Application Priority Data
Jan. 10, 2020 (DK) PA 2020 70016
Jan. 10, 2020 (DK) PA 2020 70020

(51) Int. Cl. A61K 31/44; C07D 213/80; C07D 215/54; C07D 213/82; A61K 31/47; A61P 15/08

(52) U.S. Cl. CPC A61K 31/44; C07D 213/80; C07D 215/54; C07D 213/82; A61K 31/47; A61P 15/08

(57) ABSTRACT

Some analogues (eg. 3-carbamoyl-1-(tetrahydro-2H-pyran-4-yl)pyridin-1-ium, 3-carboxy-1-isopropylpyridin-1-ium, 1-benzyl-3-carbamoylpyridin-1-ium, 3-carbamoyl-1-methylpyridin-1-ium and cyclopamine) are disclosed to treat female infertility as the compounds increase the percentage of primary follicles relative to primordial follicles compared to control samples.

### BACKGROUND

The ability to conceive a child often seems like the most natural and frequent occurrence, representing an event that is required to maintain the continuous development of our society. However, there is a large and increasing group of infertile women, who cannot be helped with the current treatment.

The only available current treatment for infertility is the standard hormone treatment. Prior to treatment, endogenous hormone levels are measured and women with high follicle stimulating hormone (FSH) levels are precluded from this treatment, as it can be lethal. The standard hormone treatment is administered mainly as intravenous administration for 5-6 days and aim to support ovarian follicles that have already been activated in the monthly ovarian cycle, and need hormonal support to complete follicle growth and maturation. Subsequently, if the procedure was successful, eggs are aspirated using ultrasound and collecting tubes, and submitted to in vitro fertilization (IVF). The fertilized egg will now be monitored using an embryoscope and if an embryo develops with high quality (as judged by morphology), the embryo will be transferred back into the uterus of the treated women. Less than 10% of women submitted to the standard hormone treatment will succeed in giving birth to an IVF-generated baby. Since the fertility treatment today is directed towards the later stages of follicle development, a large fraction of women remain infertile, presumably due to the fact that primordial (resting) follicles are not activated. Thus, there is a major unmet medical need to increase the success rate in treating female infertility.

### SUMMARY

The present invention discloses compounds identified for use in promoting follicle maturation, in particular for use in promoting the primordial to primary transition of follicles. Activation of primordial follicles is marked morphologically by the primordial-to-primary transition when the flattened granulosa cells start proliferation and become cuboidal and the oocyte grows in size.

Our invention has identified compounds that can initiate this activation of the primordial follicles. This provides a new treatment line for women who suffer from ovulation disorders resulting in infertility and who do not respond to the standard hormone treatment.

### DESCRIPTION OF DRAWINGS

FIG. 1: shows a schematic presentation of the primordial-to-primary transition. The primordial follicle consists of an oocyte almost completely surrounded by a flattened layer of somatic granulosa cells. After activation, the primordial follicle transit to a primary follicle, which is accompanied by an increase in the oocyte size as well as growth and transformation of the surrounding granulosa cells, which now become cubic and completely covers the oocyte.

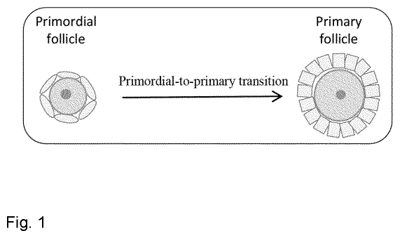

FIG. 2: shows the results of incubating ovarian sections with compound 582. (A) shows a cross section of ovaries incubated with 582. (B) is a comparison between the distribution of follicles in the different stages (primordial, primary and secondary) of ovaries treated with 582 and control. (C) shows the structure of 582.

FIG. 3: shows the results of incubating ovarian sections with compound 583. (A) shows a cross section of ovaries incubated with 583. (B) is a comparison between the distribution of follicles in the different stages (primordial, primary and secondary) of ovaries treated with 583 and control. (C) shows the structure of 583.

FIG. 4: shows the results of incubating ovarian sections with compound 797. (A) shows a cross section of ovaries incubated with 797. (B) is a comparison between the distribution of follicles in the different stages (primordial, primary and secondary) of ovaries treated with 797 and control. (C) shows the structure of 797.

FIG. 5: shows the results of incubating ovarian sections with compound 798. (A) shows a cross section of ovaries incubated with 798. (B) is a comparison between the distribution of follicles in the different stages (primordial, primary and secondary) of ovaries treated with 798 and control. (C) shows the structure of 798.

FIG. 6: is a comparison between the distribution of follicles in the different stages (primordial, primary and secondary) of ovaries treated with cyclopamine and control.

### DETAILED DESCRIPTION

The present invention relates to a compound of the formula (I)

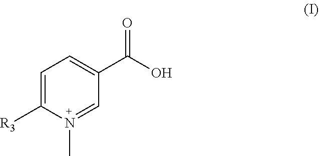

wherein R₃ is a lower alkyl or aryl.

As used herein “lower alkyl” refers to branched or straight chain acyclic alkyl group including one to about four carbon atoms. Exemplary alkyl groups include, methyl, ethyl, n-propyl, isopropyl, n-butyl, isobutyl, sec-butyl. The term “aryl” as used herein refers to a carbocyclic aromatic ring having 6 to 14 carbon atoms. Examples of aryl groups include for example phenyl and naphthyl

In one embodiment, R₃ is methyl or ethyl.

In a preferred embodiment, R₃ is methyl. The common name for that compound is 5-carboxy-1,2-dimethylpyridin-1-ium.

In another preferred embodiment, R₃ is phenyl. The common name of that compound is 5-carboxy-1-methyl-2-phenyllpyridin-1-ium.

One embodiment of the present invention relates to a compound of the formula (II)

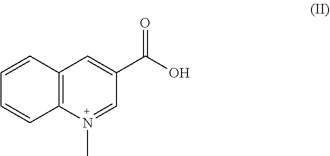

The common name of this compound is 3-carboxy-1-methylquinolin-1-ium.

In yet another embodiment the present invention relates to a compound of formula (III)

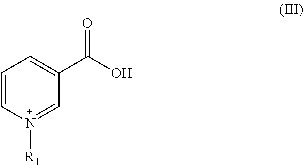

wherein R₁ is selected from the group consisting of: oxocyclohexanyl, oxocyclopentanyl and oxocyclobutanyl. In a preferred embodiment R₁ is oxocyclohexanyl. The common name of this compound is 3-carboxy-1-(tetrahydro-2H-pyran-4-yl))pyridin-1-ium. In the present application 3-carboxy-1-(tetrahydro-2H-pyran-4-yl))pyridin-1-ium is referred to by the number 798.

Another embodiment of the present invention relates to a compound of formula (IV)

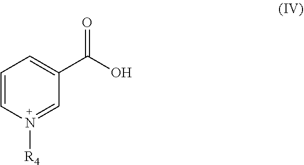

wherein R₄ is a lower alkyl, an amide, a phenyl or a benzyl for use in the treatment of an ovulation disorder.

As used herein “lower alkyl” refers to branched or straight chain acyclic alkyl group including one to about four carbon atoms. Exemplary alkyl groups include, methyl, ethyl, n-propyl, isopropyl, cyclopropyl, n-butyl, isobutyl, sec-butyl. In a further embodiment said lower alkyl is: ethyl, propyl or isopropyl.

I one embodiment said, lower alkyl is methyl, ethyl, propyl or isopropyl. In a preferred embodiment R₄ is isopropyl for use in the treatment of an ovulation disorder. The common name of that compound is 3-carboxy-1-ispropylpyridin-1-ium. In the present application 3-carboxy-1-ispropylpyridin-1-ium is referred to by the number 797.

In another embodiment, R₄ is ethanamide. The common name of that compound is 1-(2-amino-2-oxoethyl)-3-carboxypyridin-1-ium.

In another embodiment, R₄ is phenyl. The common name of that compound is 3-carboxy-1-phenylpyridin-1-ium.

In yet another embodiment, R₄ is benzyl. The common name of that compound is 1-benzyl-3-carboxypyridin-1-ium.

In yet another embodiment the present invention relates to a compound of formula (V):

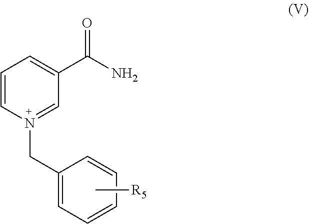

wherein R₅: H, F, Cl or a methoxy group for use in the treatment of an ovulation disorder.

In a preferred embodiment R₅ is H. The common name for this compound is 1-benzyl-3-carbamoylpyridin-1-ium. In the present application 1-benzyl-3-carbamoylpyridin-1-ium is referred to by the number 582. In another embodiment R₅ is F in the ortho position. In a further embodiment R₅ is Cl in the meta or para position. In yet another embodiment R₅ is a methoxy group in the meta position.

In a further embodiment the present invention relates to a compound of formula (VI):

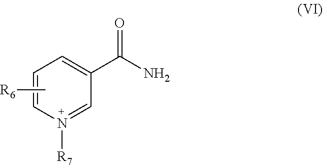

wherein R₇ is a lower alkyl and R₆ is H or methyl for use in the treatment of an ovulation disorder. In one embodiment said, lower alkyl is methyl, ethyl, propyl or isopropyl. In a preferred embodiment R₇ is isopropyl.

In another preferred embodiment R₇ is methyl and R₆ is H. The common name for this compound is 3-carbamoyl-1-methylpyridin-1-ium. In the present application 3-carbamoyl-1-methylpyridin-1-ium is referred to by the number 583. In another embodiment R₇ is ethyl and R₆ is H. In a further embodiment R₇ is methyl and R₆ is methyl in the ortho position. In yet another embodiment R₇ is methyl and R₆ is methyl in the para position.

The compounds above herein can be prepared via the following two general synthetic routes:

Route 1:

This is an alkylation at the nitrogen using alkyl halides. X can be I or Br. R represents any substituent on the pyridine moiety.

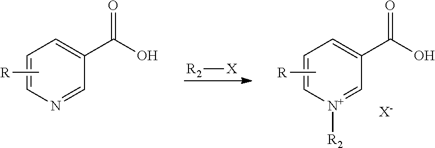

Route 2:

Route 2 is a Zincke reaction. R represents any substiuent on the pyridine moiety and R₂ represents the substiuent to be added to the nitrogen of the pyridine.

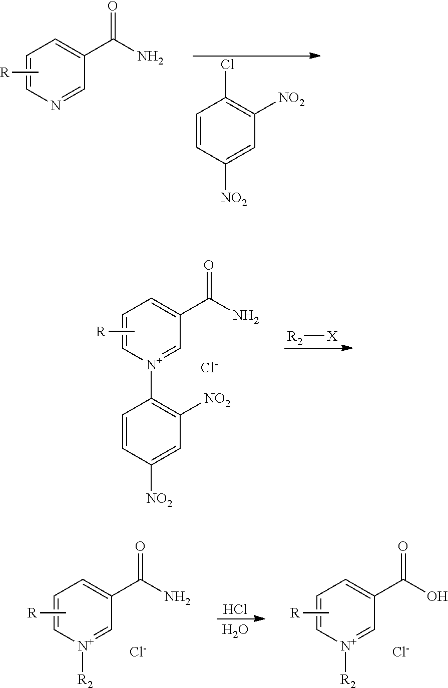

In an alternative embodiment the present invention relates to cyclopamine for use in the treatment of an ovulation disorder. Cyclopamine is represented by the structure of formula VII below:

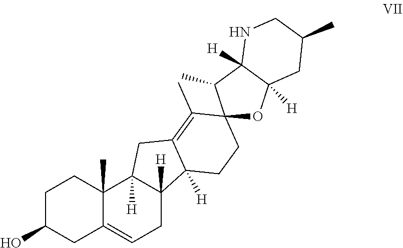

Cyclopamine is a naturally occurring chemical that is isolated from the corn lily.

Several compounds according to the present invention comprise a positive charge. Pharmaceutically acceptable salts of compounds suited for use in accordance with the invention may therefore be obtained by inclusion of a negatively charged ion. Examples of such negatively charged ions are: chloride, iodide and bromide. Other negatively charged ions may be used to obtain a pharmaceutically acceptable salt of the compounds according to the present disclosure.

As described in example 1, none of the tested compounds display toxicity against mammalian cells. Thus in one embodiment, the compounds according to the present invention can be used as a medicament.

In one embodiment of the invention, a method of treating a medical condition comprising administering a therapeutically effective amount of the compounds described or pharmaceutically acceptable salts thereof to a human or animal in need thereof is provided.

In another embodiment, the compounds according to the present invention can be used for the manufacture of a medicament.

Furthermore, the compounds according to the present invention promote the maturation of follicles by stimulating the transition of follicles from primordial to primary follicles. The compounds according to the present invention can be used in vivo wherein they are administered to a female individual in need thereof. Thus, in one embodiment the compounds as described herein are for use in promoting the maturation of follicles in vivo.

The compounds can also be used in vitro wherein they are added in vitro to follicles. Thus, in another embodiment the compounds as described herein are for use in promoting the maturation of follicles in vitro.

In a preferred embodiment, said follicles are mammalian follicles. Mammalian follicles can stem from any mammal such as livestock for example horses, pigs or cows. In a preferred embodiment, the mammal is a cow. In a preferred embodiment, the mammalian is a human.

Many cases of female infertility are caused by ovulation disorders. Problems with the regulation of reproductive hormones by the hypothalamus or the pituitary gland, or problems in the ovary, can cause ovulation disorders. As current standard fertility treatment is directed towards the later stages of follicle development, the standard hormone therapy is very effective towards women that can produce secondary follicles.

Our research has shown, that most ovulation disorders are caused by an absence of the initial activation of the primordial follicles or activation of too few, which results in no production of primary and secondary follicles. The compounds of the current invention promote the transition of follicles from primordial to primary follicles. Our invention therefore suggests that a new line of treatment would be to target the primordial follicles, which is required to start the ovarian cycle to generate a mature and fertile egg.

In one embodiment, the compounds described herein are for use in the treatment of infertility in women not responding to standard current hormone treatment. In a preferred embodiment that is due to an ovulation disorder. Thus, in one embodiment the present invention relates to the compounds described herein for use in the treatment of an ovulation disorder.

In one embodiment said ovulation disorder is selected from the group consisting of premature ovarian insufficiency (POI) and premature menopause.

POI is an ovulation disorder causing infertility. POI, sometimes referred to as premature menopause, occurs when a woman's ovaries fail before she is 40 years of age. POI can be caused by a genetic abnormality (such as Turner syndrome or Fragile X, autoimmune diseases or exposure to medicines or radiotherapy). However, idiopathic POI is a relatively poorly understood disease that causes infertility and manifests itself with a multitude of additional health implications such as osteoporosis due to lack of sufficient amounts of estrogen, various autoimmune diseases, mental health diseases (increased risk of suicide), hypothyroidism and heart diseases. POI often presents in women in their early twenties, where premature menopause-like symptoms develop. Menopause represents a major hormonal change, characterized by a decline in oestrogen and progesterone levels and cessation of female reproductive function as the ovarian reserve is exhausted.

Certain exposures, such as chemotherapy or pelvic radiation therapy, and certain medical conditions may cause POI, however, the cause is often unexplained.

Whilst it is possible for the compounds of the present invention to be administered alone, it is preferred to present them in the form of a pharmaceutical formulation.

An aspect of the present invention relates to a pharmaceutical composition comprising at least one compound as defined herein for use in the treatment of an ovulation disorder in a female mammal, preferably the female mammal is a female human.

It is preferred that the pharmaceutical composition further comprises at least one pharmaceutically acceptable carrier.

The pharmaceutically acceptable carriers can be either solid or liquid. Solid form preparations include powders, tablets, pills, capsules, cachets, suppositories, and dispersible granules. A solid carrier can be one or more excipients which may also act as diluents, flavoring agents, solubilizers, lubricants, suspending agents, binders, preservatives, wetting agents, tablet disintegrating agents, or an encapsulating material.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

In powders, the carrier is a finely divided solid which is a mixture with the finely divided active component. In tablets, the active component is mixed with the carrier having the necessary binding capacity in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like.

Liquid form of the pharmaceutically acceptable carrier allow for parental administration and may be presented in unit-dose or multi-dose sealed containers, such as ampoules, vials, pre-filled syringes, infusion bags, optionally with an added preservative or it can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

Other forms suitable for oral administration may include liquid form preparations including emulsions, syrups, elixirs, aqueous solutions, aqueous suspensions.

Preferably, the formulation will comprise about 0.5% to 75% by weight of the active ingredient(s) with the remainder consisting of suitable pharmaceutical excipients as described herein.

Pharmaceutically acceptable salts of the instant compounds, where they can be prepared, are also intended to be covered by this invention. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids and arylsulphonic acid.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. Suitable solvents for the preparation of an oily solution include glycerol, diluted alcohol and propylene glycol.

Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavors, stabilizing and thickening agents. Aqueous suspensions can be prepared by dispersing the finely divided active component in water with viscous material, such as natural or synthetic gums, resins, methylcellulose, sodium carboxymethylcellulose, and other well-known suspending agents.

In one embodiment the pharmaceutical composition comprises an additional active agent. The pharmaceutical composition may also comprise a combination of the compounds as described herein.

As described herein administration forms include but are not limited to oral, parental, enteral, rectal or buccal administration.

In one embodiment the pharmaceutical composition is administered or adapted for administration enterally, parenterally or as part of a sustained release implant. The parenteral administration may for example be intravenous, subcutaneous, intramuscular, intracranial or intraperitoneal.

In a preferred embodiment the pharmaceutical composition is administered by or adapted for injection. In a preferred embodiment the pharmaceutical composition is administered by or adapted for injection into the ovaries.

In one embodiment the compound as described herein is to be administered in a dosage of from 1 μg/kg-30,000 μg/kg body weight, such as 1 μg/kg-7,500 μg/kg, such as 1 μg/kg-5,000 μg/kg, such as 1 μg/kg-2,000 μg/kg, such as 1 μg/kg-1 ,000 μg/kg, such as 1 μg/kg-700 μg/kg, such as 5 μg/kg-500 μg/kg, such as 10 μg/kg to 100 μg/kg bodyweight. In another embodiment the compound as described herein is to be administered in a dosage of from 1 μg/kg-1,000 μg/kg body weight, such as 1 μg/kg-500 μg/kg, such as 1 μg/kg-250 μg/kg, such as 1 μg/kg-100 μg/kg, such as 1 μg/kg-50 μg/kg, such as 1 μg/kg to 10 μg/kg bodyweight. In yet another embodiment the compound as described herein is to be administered in a dosage of from 10 μg/kg-30,000 μg/kg body weight, such as 10 μg/kg-7,500 μg/kg, such as 10 μg/kg-5,000 μg/kg, such as 10 μg/kg-2,000 μg/kg, such as 10 μg/kg-1,000 μg/kg, such as 10 μg/kg-700 μg/kg, such as 10 μg/kg-500 μg/kg, such as 10 μg/kg to 100 μg/kg bodyweight.

The pharmaceutical composition of the present invention may also be administered in vitro to primordial follicles from said female individual. Thus, primordial follicles can be taken from an individual in need thereof, treated in vitro with at least one compound of the present invention and re-inserted into said female individual.

In one embodiment said female individual is a female mammal. Preferably, said female mammal is a female human.

### EXAMPLES

### Example A: Synthesis of 5-carboxy-1,2-dimethylpyridin-1-ium iodide

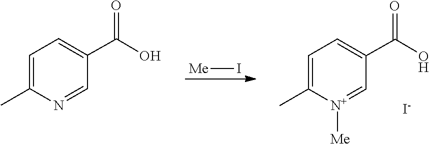

6-methylnicotinic acid, N,N-dimethylformamide and iodomethane is placed in a vial. This is sealed and covered in foil to exclude light and the mixture is stirred for 18 hours at 50° C. The reaction mixture is concentrated under vacuum to give an oil. The crude product is dissolved in methanol and precipitated as an oil by addition of diethyl ether. The ether layer is removed and the residue is dried in vacuo. The crude material is recrystallised from water to give a solid.

1H NMR assignment: (400 MHz, DMSO-d6) δ 9.50 (d, J=1.4 Hz, 1H), 8.81 (dd, J=8.2, 1.8 Hz, 1H), 8.13 (d, J=8.2 Hz, 1H), 4.29 (s, 3H), 2.83 (s, 3H)

### Example B: Synthesis of 5-carboxy-1-methyl-2-phenyllpyridin-1-ium iodide

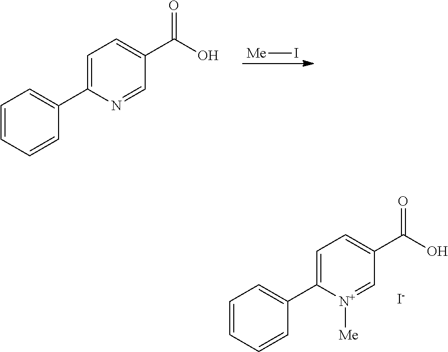 [SMILES: O=C(O)c1ccc(-c2ccccc2)nc1 ; CI ; C[n+]1cc(C(=O)O)ccc1-c1ccccc1 ; [I-]]

6-phenylnicotinic acid, N,N-dimethylformamide and iodomethane is placed in a vial. This is sealed and covered in foil to exclude light and the mixture is stirred for 18 hours at 50° C. The reaction mixture is concentrated under vacuum to give an oil. The crude product is dissolved in methanol and precipitated as an oil by addition of diethyl ether. The ether layer is removed and the residue is dried in vacuo. The crude material is recrystallised from water to give a solid.

¹H NMR assignment: (400 MHz, DMSO-d6) δ 9.68 (d, J=1.8 Hz, 1H), 8.95 (dd, J=8.2, 1.8 Hz, 1H), 8.17 (d, J=8.2 Hz, 1H), 7.73-7.65 (m, 5H), 4.19 (s, 3H)

### Example C: Synthesis of 3-carboxy-1-methylquinolin-1-ium iodide

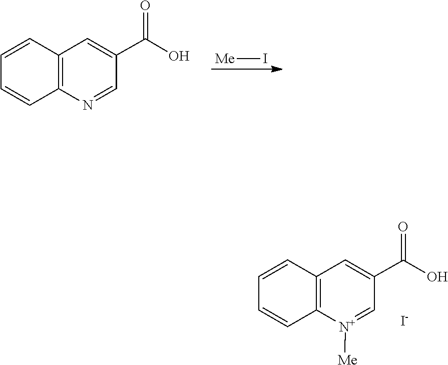 [SMILES: O=C(O)c1cnc2ccccc2c1 ; CI ; C[n+]1cc(C(=O)O)cc2ccccc21 ; [I-]]

3-Quinolinecarboxylic acid, N,N-dimethylformamide and iodomethane is placed in a vial. This is sealed and covered in foil to exclude light and the mixture is stirred for 18 hours at 50° C. The reaction mixture is concentrated under vacuum to give an oil. The crude product is dissolved in methanol and precipitated as an oil by addition of diethyl ether. The ether layer is removed and the residue is dried in vacuo. The crude material is recrystallised from water to give a solid.

¹H NMR assignment: (400 MHz, DMSO-d6) δ 9.96 (d, J=0.9 Hz, 1H), 9.79 (br.s, 1H), 8.66 (dd, J=8.2, 1.4 Hz, 1H), 8.56 (d, J=8.7 Hz, 1H), 8.42-8.37 (m, 1H), 8.15 -8.11 (m, 1H), 4.70 (s, 3H)

### Example D: Synthesis of 3-carbamoyl-1-(tetrahydro-2H-pyran-4-yl)pyridin-1-ium (798)

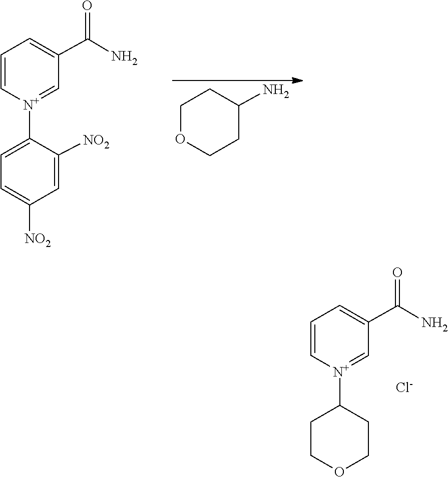 [SMILES: NC(=O)c1ccc[n+](-c2ccc([N+](=O)[O-])cc2[N+](=O)[O-])c1 ; NC1CCOCC1 ; NC(=O)c1ccc[n+](C2CCOCC2)c1 ; [Cl-]]

A solution of 4-aminotetrahydropyran (125 mg, 1.23 mmol) in methanol (2.0 mL) was treated with 1-(2,4-dinitrophenyl)pyridin-1-ium-3-carboxamidm chloride (200 mg, 0.616 mmol) and the mixture was stirred for 18 hours at rt. The reaction mixture was passed through a frit, washing with MeOH (4 mL). The filtrate was diluted with ether (10 mL) and the resulting precipitate was filtered under vacuum to give the title compound as a yellow solid (80 mg, 54%).

¹H NMR assignment: (400 MHz, DMSO-d6) δ 9.56 (s, 1H), 9.42-9.41 (m, 1H), 8.96 (dt, J=8.2, 1.4 Hz, 1H), 8.28 (dd, J=7.8, 6.4 Hz, 1H), 7.39-7.14 (apparent 1:1:1 triplet, ammonium chloride, ˜0.73 H total), 5.16 (tt, J=11.9, 4.1 Hz, 1H), 4.07 (dd, J=11.0, 4.1 Hz, 2H), 3.46 (td, J=11.7, 2.0 Hz, 2H), 2.26-2.10 (m, 4H).

### Example E: Synthesis of 3-carboxy-1-phenylpyridin-1-ium chloride:

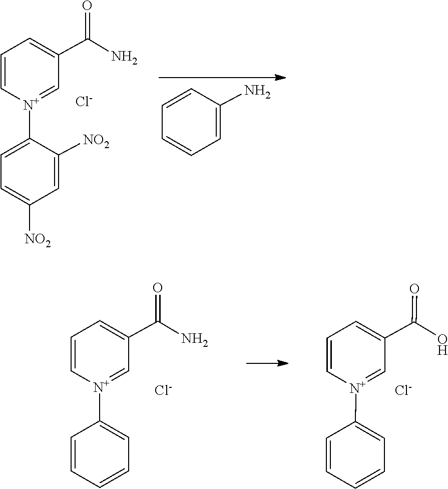

1-(2,4-dinitrophenyl)pyridin-1-ium-3-carboxamide chloride (0.20 g, 0.616 mmol) was dissolved in anhydrous methanol (2.0 mL) then aniline (0.16 mL, 1.24 mmol) was added. The reaction mixture was maintained overnight then filtered through a sintered tube and the solid was washed with methanol (3×1 mL). The filtrate was diluted with ether (˜30 mL), causing a precipitate to form, and the mixture was filtered through a sintered tube again. The solid was washed with ether then briefly sucked dry to give 1-phenylpyridin-1-ium-3-carboxamide chloride as a yellow solid (105 mg, 73%).

1-phenylpyridin-1-ium-3-carboxamide chloride (88 mg, 0.375 mmol) and concentrated hydrochloric acid (32%) (1.0 mL, 10.2 mmol) were placed in an Ace pressure tube, which was purged with nitrogen, sealed and placed behind a blast shield then heated to 60° C. overnight.

The reaction mixture was diluted with dioxane (5 mL) then concentrated to dryness in vacuo. The residue was azeotroped with toluene (×2). The residue was triturated with isopropanol, then dissolved in ethanol, filtered through a cotton wool plug and precipitated by addition of ether (repeated twice) to give the title compound as a white solid (29 mg, 33%).

¹H NMR assignment: (400 MHz, DMSO-d6) δ 9.54 (m, 1H), 9.49 (dt, J=6.0, 1.4 Hz, 1H), 9.12 (dt, J=7.8, 1.4 Hz, 1H), 8.38 (dd, J=8.0, 6.2 Hz, 1H), 7.94-7.89 (m, 2H), 7.76-7.72 (m, 3H)

### Example F: Synthesis of 1-benzyl-3-carboxypyridin-1-ium bromide:

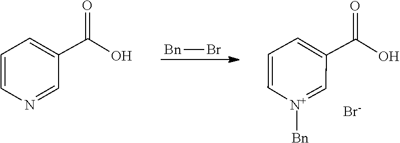 [SMILES: O=C(O)c1cccnc1 ; BrCc1ccccc1 ; O=C(O)c1ccc[n+](Cc2ccccc2)c1 ; [Br-]]

Nicotinic acid (100 mg, 0.812 mmol), N,N-dimethylformamide (1 mL) and benzyl bromide (0.11 mL, 0.894 mmol) were placed in a vial. This was sealed and covered in foil to exclude light and the mixture was stirred for 18 hours at 50° C. The reaction mixture was concentrated under vacuum to give a pale yellow oil. The crude product was dissolved in methanol and precipitated as an oil by addition of diethyl ether. The ether layer was removed and the residue was dried in vacuo. The crude material was recrystallised from water to give a white solid (110 mg, 46%).

¹H NMR assignment: (400 MHz, DMSO-D6) δ 9.54 (1H, s), 9.23-9.15 (1H, m), 8.91-8.83 (1H, m), 8.20-8.12 (1H, m), 7.57-7.50 (2H, m), 7.48-7.38 (3H, m), 5.91 (2H, s)

### Example G: Synthesis of 3-carboxy-1-isopropylpyridin-1-ium (797)

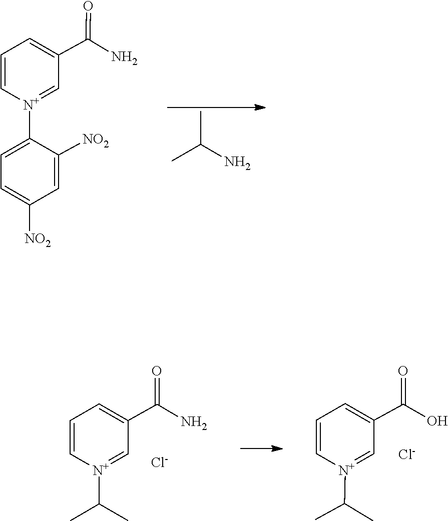

1-(2,4-dinitrophenyl)pyridin-1-ium-3-carboxamide chloride is dissolved in anhydrous methanol then 2-aminopropane is added. The reaction mixture is maintained overnight then filtered through a sintered tube and the solid is washed with methanol. The filtrate is diluted with ether, causing a precipitate to form, and the mixture is filtered through a sintered tube again. The solid is washed with ether then briefly sucked dry to 3-carbamoyl-1-isopropyl pyridin-1-ium chloride.

3-carbamoyl-1-isopropyl pyridin-1-ium chloride and concentrated hydrochloric acid (32%) is placed in an Ace pressure tube, which is purged with nitrogen, sealed and placed behind a blast shield then heated to 60° C. overnight.

The reaction mixture is diluted with dioxane then concentrated to dryness in vacuo. The residue is azeotroped with toluene (×2). The residue is triturated with isopropanol, then dissolved in ethanol, filtered through a cotton wool plug and precipitated by addition of ether (repeated twice) to give the title compound.

¹H NMR assignment: (400 Hz, DMSO-d6) δ 9.55 (s, 1H), 9.40-9.38 (m, 1H), 8.94 (dt, J=8.2, 1.4 Hz, 1H), 8.27 (dd, J=8.2, 6.0 Hz, 1H), 7.39-7.11 (m, approx 1:1:1 triplet, ammonium ion, ˜4H), 5.18 (apparent quintet, outermost lines of expected septet not distinguishable, J=6.8 Hz, 1H), 1.63 (d, J=6.4 Hz, 6H)

### Example H: Synthesis of 1-(2-amino-2-oxo-ethyl)pyridin-1-ium-3-carboxylic acid bromide:

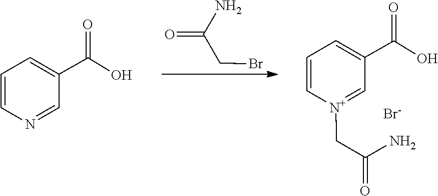 [SMILES: O=C(O)c1cccnc1 ; NC(=O)CBr ; NC(=O)C[n+]1cccc(C(=O)O)c1 ; [Br-]]

Nicotinic acid, N,N-dimethylformamide and 2-bromoacetamide is placed in a vial. This is sealed and covered in foil to exclude light and the mixture was stirred for 18 hours at 50° C. The reaction mixture is concentrated under vacuum to give an oil. The crude product is dissolved in methanol and precipitated as an oil by addition of diethyl ether. The ether layer is removed and the residue is dried in vacuo. The crude material is recrystallised from water to give a white solid.

¹H NMR assignment: (400 MHz, DMSO-D6) δ 9.36 (1H, s), 9.02-8.97 (1H, m), 8.94-8.88 (1H, m), 8.20-8.13 (1H, m), 8.04 (1H, s), 7.70 (1H, s), 5.46 (2H, s)

### Example I: Synthesis of 1-benzyl-3-carbamoylpyridin-1-ium (582) bromide

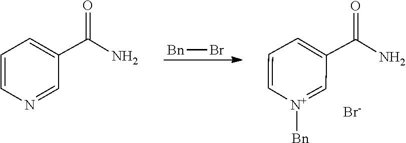 [SMILES: NC(=O)c1cccnc1 ; BrCc1ccccc1 ; NC(=O)c1ccc[n+](Cc2ccccc2)c1 ; [Br-]]

Nicotinamide, N,N-dimethylformamide and benzylbromide is placed in a vial. This is sealed and covered in foil to exclude light and the mixture was stirred for 18 hours at 50° C. The reaction mixture is concentrated under vacuum to give an oil. The crude product is dissolved in methanol and precipitated as an oil by addition of diethyl ether. The ether layer is removed and the residue is dried in vacuo. The crude material is recrystallised from water to give a white solid.

¹H NMR assignment: (400 MHz, DMSO-D6) δ 9.63 (1H, s), 9.32-9.27 (1H, m), 9.00-8.93 (1H, m), 8.60 (1H, s), 8.33-8.25 (1H, m), 8.19 (1H, s), 7.61-7.53 (2H, m), 7.51-7.39 (3H, m), 5.92 (2H, s).

### Example J: Synthesis of 3-carbamoyl-1-methylpyridin-1-ium (583) iodide

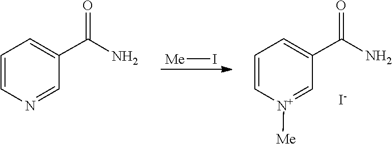 [SMILES: NC(=O)c1cccnc1 ; CI ; C[n+]1cccc(C(N)=O)c1 ; [I-]]

Nicotinamide, N,N-dimethylformamide and iodomethane is placed in a vial. This is sealed and covered in foil to exclude light and the mixture was stirred for 18 hours at 50° C. The reaction mixture is concentrated under vacuum to give an oil. The crude product is dissolved in methanol and precipitated as an oil by addition of diethyl ether. The ether layer is removed and the residue is dried in vacuo. The crude material is recrystallised from water to give a white solid.

¹H NMR assignment: (400 MHz, DMSO-D6) δ 9.36 (1H, s), 9.02-8.97 (1H, m), 8.94-8.88 (1H, m), 8.20-8.13 (1H, m), 8.04 (1H, s), 7.70 (1H, s), 5.46 (2H, s).

### Example 1—Toxicity Assay

ID8 Mouse Ovarian Surface Epithelial Cell Line (Milipore, SCC145), were plated in High Glucose DMEM media (Sigma Cat. No. D6429), 4% FBS (Cat. No. ES-009-C), 5 μg/mL insulin, 5 μg/mL transferrin and 5 ng/mL sodium selenite (1× ITS; Gibco Cat. No. 41400045) and 1× Penicillin-Streptomycin Solution (Thermo Fisher Cat. No. 15140122) at 150000 cell density in 35 mm dish. Immediately, 1 μM compound was added to the media and cells were grown for 2 days and morphology checked using light microscopy.

The compounds 3-carbamoyl-1-(tetrahydro-2H-pyran-4-yl)pyridin-1-ium (798), 3-carboxy-1-methylquinolin-1-ium (797), 1-benzyl-3-carbamoylpyridin-1-ium (582), 3-carbamoyl-1-methylpyridin-1-ium (583) and cyclopamine were tested in the toxicity assay and were all found not to alter the morphology of the cells nor to be toxic to the grown cells.

### Example 2—Evaluation of Follicle Numbers After Treatment with Compounds 798, 797, 582, 583 and Cyclopamine

### Animals

C57BL/6j×CBA F1 hybrid mice (Janvier Labs, France) were housed and bred in the animal facilities at Department of Biomedicine, Aarhus University. Animals were housed in a 12:12 h controlled light-dark environment and were provided with food and water ad libitum. 7-day-old female pups were used for the study.

### Isolation of Ovaries and Organ Culture

Pups were sacrificed and ovaries were excised. Using a stereomicroscope MZ75 (Leica Microsystems, Germany), excess tissue was removed. During the dissection, ovaries were kept at 37° C. in culture medium: αMEM (Thermo Fisher) supplemented with 10% FBS (Thermo Fisher), 100 mIU/mL of FSH (Sigma-Aldrich), 100 IU/mL penicillin (Thermo Fisher), 100 μg/mL streptomycin (Thermo Fisher) and 1% Insulin-Transferrin-Selenium (Thermo Fisher). Isolated ovaries were transferred into well inserts (PET membrane ThinCert, 0.4 μm-pore size; Greiner bio-one) in 24-well plates (Tissue culture treated, 20cell culture plates; Costar). 200-300 μL of culture medium was added to the well below the insert and up to two ovaries were placed on the membrane of each insert. The culture medium was supplemented with 0.5 μM and 1 μM of compound for compounds 582 and 583 or 25 μM of compound for compounds 798 and 798 and finally with 0.5 μM of compound for cyclopamine. Up to six wells in the 24-well plate were used for inserts. Sterile dH2O was added to the rest of the wells to ensure humidity. The ovaries were cultured at 37° C., 5% CO₂ for four days. 150 μL of medium was replaced every other day with fresh culture medium.

### Histological Analysis and Follicle Counting

Ovaries kept in organ culture for four days were fixed for 24 hours in 4% paraformaldehyde solution at 4° C. After fixation, ovaries were dehydrated in ethanol series using 70%, 96% and 99.9% ethanol. Xylene was used as clearing agent before the ovaries were infiltrated in paraffin wax. 5 μm-sections of samples in paraffin were cut using a microtome (Cut 6062, SLEE medical, Germany). Paraffin sections were mounted on glass slides, paraffin was melted at 60° C., and the samples were stained with hematoxylin and eosin (using standard protocols).The samples were deparaffinized by incubating in xylene for 2×15 min. and subsequently rehydrated in series of ethanol; 3×2 min. in 99.9% ethanol, 2 min. in 96% ethanol, and 2 min. in 70% ethanol. The samples were then rinsed in dH2O, stained in hematoxylin for 40 sec., rinsed in dH2O for 5 min., and stained in eosin for 46 sec. The samples were dehydrated in ethanol: 2×2 min. in 96% ethanol, 2×2 min in 99.9% ethanol, and finally cleared up in xylene for at least 30 min before mounting the samples using Eukitt mounting medium (Sigma-Aldrich) and cover glass. The number of follicles at each developmental stage was counted using an inverted research microscope (DMI4000B, Leica Microsystems, Germany). The follicles of every 3^{rd} to 4^{th} section of each ovary were counted and the distribution of follicles in the different stages in percentage was determined. Every treatment was repeated on at least three biological repeats. Only follicles with a visible oocyte nucleus was counted. Follicles were classified as either primordial, primary or secondary. Briefly, primordial follicles consist of an oocyte encapsulated by flattened, squamous granulosa cells. Primary follicles are oocytes encapsulated by one layer of cuboidal granulosa cells, and secondary follicles consist of oocytes encapsulated by more than one layer of cuboidal granulosa cells.

### Statistical Analysis

The percentages of follicles in the different stages from each biological repeat were averaged. When comparing two groups, an unpaired t-test was performed. One-way

ANOVA followed by determination of statistical significance using the Holm-Sidak method was performed when comparing more than two groups. Groups were considered significantly different if P≤0.05. Statistics were calculated with the help of GraphPad Prism (version 7.00 GraphPad Software, La Jolla, Calif., USA).

### Results

The effect of incubating ovaries with compounds 3-carbamoyl-1-(tetrahydro-2H-pyran-4-yl)pyridin-1-ium (798), 3-carboxy-1-methylquinolin-1-ium (797), 1-benzyl-3-carbamoylpyridin-1-ium (582), 3-carbamoyl-1-methylpyridin-1-ium (583) and cyclopamine were evaluated as described above.

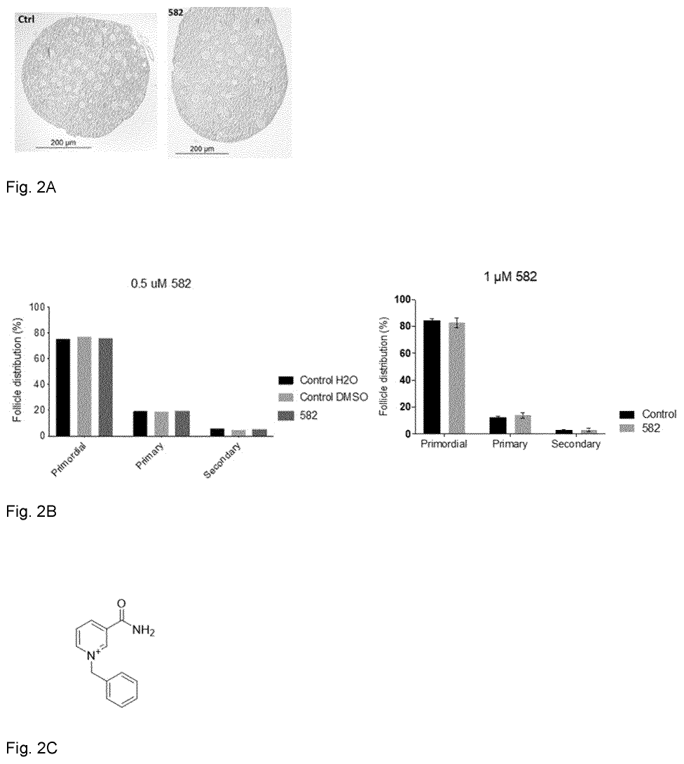

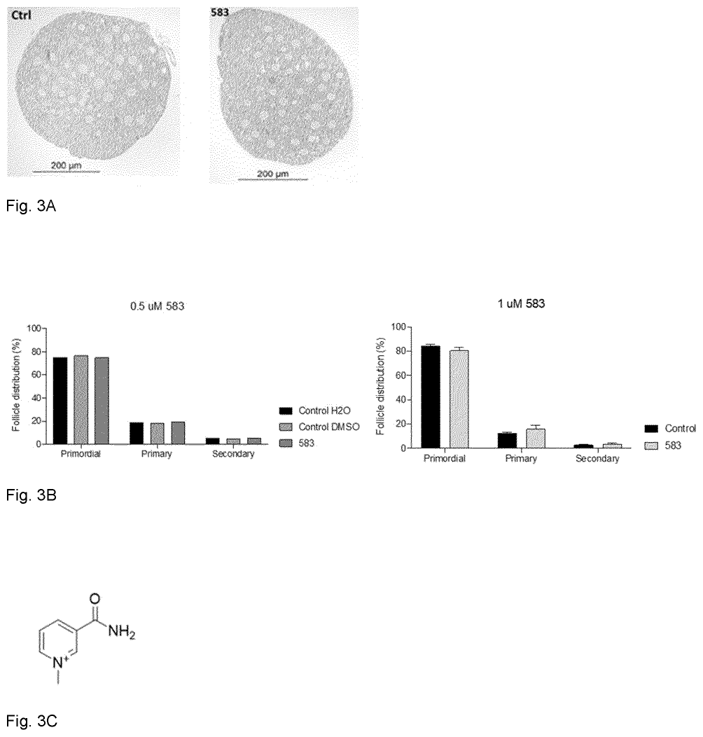

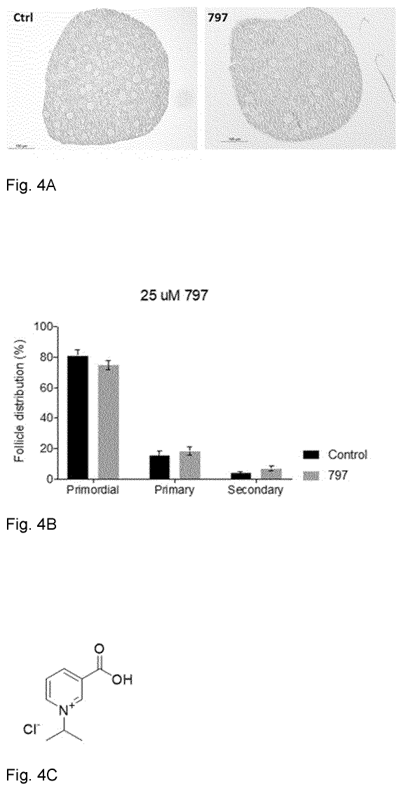

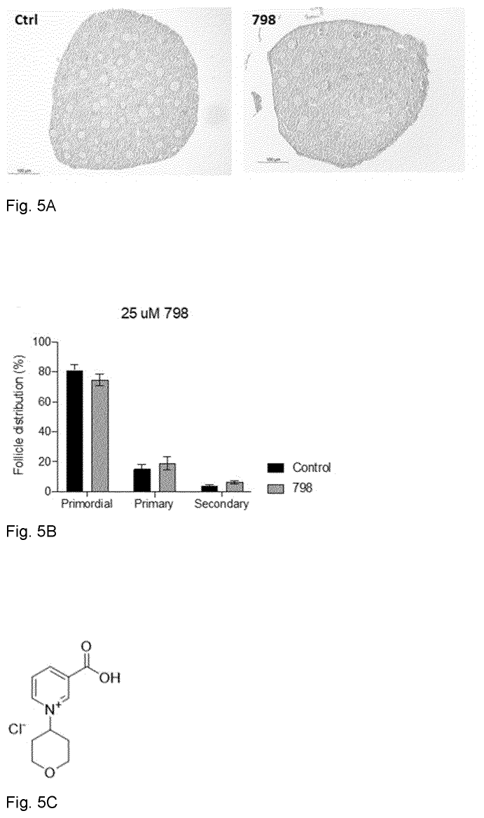

The ovarian sections were visually inspected after incubation with the compounds 798, 797, 582 and 583 (FIG. 2A-5A). It was found that the overall appearance of the ovaries in culture was healthy and the size was normal, no shrinkage was observed.

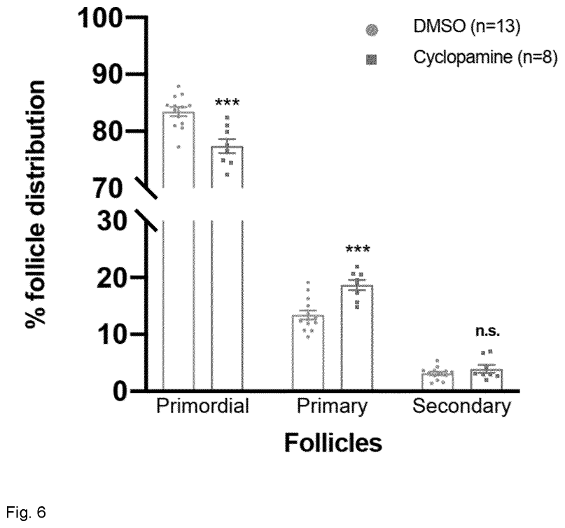

Furthermore, the morphology of the follicles looked healthy as the growth of these was normal in regards to the size of the cells and the contact between cells. Oocytes and granulosa cells were not separated. Finally, it was found that the activation of follicles, that is the conversion from primordial to primary and secondary follicles was notable for the samples where compound had been added compared to samples, where no compound was added (FIG. 2B-5B and FIG. 6).

1. A compound of formula (III) 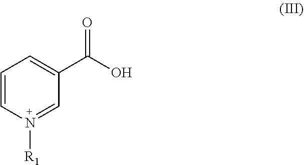
or a pharmaceutically acceptable salt thereof; wherein R₁ is selected from the group consisting of: oxocyclohexanyl, oxocyclopentanyl and oxocyclobutanyl.

2. The compound according to claim 1, wherein R₁ is oxocyclohexanyl.

3. A compound of the formula (I) or a pharmaceutically acceptable salt thereof 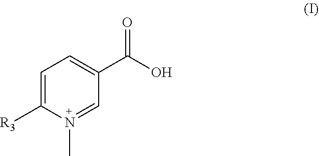
wherein R₃ is a lower alkyl or aryl.

4. The compound according to claim 3 wherein said lower alkyl is methyl or ethyl.

5. The compound according to claim 3 wherein R₃ is methyl.

6. The compound according to claim 3 wherein said R₃ is phenyl.

7. A compound of the formula (II) or a pharmaceutically acceptable salt thereof

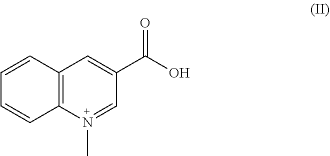

8. The compound according to any of the preceding claims for use as a medicament.

9. The compound according to any of the preceding claims for use in promoting follicle maturation

10. The compound according to any of the preceding claims for use in the treatment of an ovulation disorder.

11. The compound according to claim 10, wherein said ovulation disorder is selected from the group consisting of premature ovarian insufficiency (POI) and premature menopause.

12. A method of treating a medical condition comprising administering a therapeutically effective amount of the compound according to claims 1-7 or pharmaceutically acceptable salts thereof to a human or animal in need thereof.

13. The method according to claim 12, wherein said medical condition is an ovulation disorder.

14. The method according to claim 13, wherein said ovulation disorder is selected from the group consisting of premature ovarian insufficiency (POI) and premature menopause.

15. The use of the compound according to claims 1-7 for the manufacture of a medicament.

16. The use of the compound according to claim 1-7 for the manufacture of a medicament for use in the treatment of an ovulation disorder.

17. The use of the compound according to claim 16, wherein said ovulation disorder is selected from the group consisting of premature ovarian insufficiency (POI) and premature menopause.

18. A compound of the formula (IV) or a pharmaceutically acceptable salt thereof 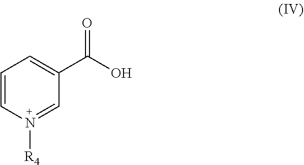
wherein R₄ is a lower alkyl, an amide, a phenyl or a benzyl for use in the treatment of an ovulation disorder.

19. The compound according to claim 18 wherein said lower alkyl is ethyl, propyl or isopropyl.

20. The compound according to claims 18 and 19 wherein R₄ is isopropyl.

21. The compound according to claim 18 wherein R₄ is ethanamide.

22. The compound according to claim 18 wherein R₄ is phenyl.

23. The compound according to claim 18 wherein R₄ is benzyl.

24. A compound of formula (V) or a pharmaceutically acceptable salt thereof 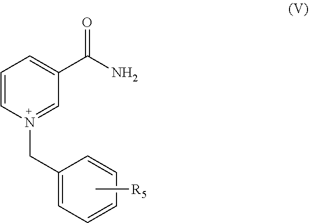
wherein R₅ is H, F, Cl or a methoxy group for use in the treatment of an ovulation disorder.

25. The compound according to claim 24 wherein R₅ is H.

26. The compound according to claim 24 wherein R₅ is F in the ortho position.

27. The compound according to claim 24 wherein R₅ is Cl in the meta or para position.

28. The compound according to claim 24 wherein R₅ is a methoxy group in the meta position.

29. A compound of formula (VI) or a pharmaceutically acceptable salt thereof 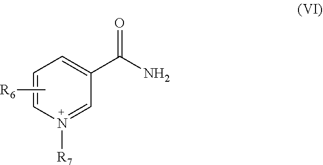
wherein R₇ is a lower alkyl and R₆ is H or methyl for use in the treatment of an ovulation disorder.

30. The compound according to claim 29 wherein R₇ is methyl, ethyl, propyl or isopropyl.

31. The compound according to claim 29 wherein R₇ is isopropyl.

32. The compound according to claim 29 wherein R₇ is methyl and R₆ is H.

33. The compound according to claim 29 wherein R₇ is ethyl and R₆ is H.

34. The compound according to claim 29 wherein R₇ is methyl and R₆ is methyl in the ortho position.

35. The compound according to claim 29 wherein R₇ is methyl and R₆ is methyl in the para position.

36. The compound cyclopamine for use in the treatment of an ovulation disorder.

37. The compound according to claims 18-36 for use in promoting follicle maturation.

38. The compound according to claims 18-36, wherein said ovulation disorder is selected from the group consisting of premature ovarian insufficiency (POI) and premature menopause.

39. A method of treating an ovulation disorder comprising administering a therapeutically effective amount of the compound according to claims 18-36 or pharmaceutically acceptable salts thereof to a human or animal in need thereof.

40. The method according to claim 39, wherein said ovulation disorder is selected from the group consisting of premature ovarian insufficiency (POI) and premature menopause.

41. The use of the compound according to claims 18-36 for the manufacture of a medicament for the treatment of an ovulation disorder.

42. The use of the compound according to claim 41, wherein said ovulation disorder is selected from the group consisting of premature ovarian insufficiency (POI) and premature menopause.

43. A pharmaceutical composition comprising the compound according to any of the preceding claims for use in the treatment of an ovulation disorder in a female individual.

44. The pharmaceutical composition according to claim 43, further comprising at least one pharmaceutically acceptable carrier.

45. The pharmaceutical composition according to claims 43 and 44, further comprising an additional active agent.
